(19) United States
(12) Patent Application Publication
(10) Pub. No.: US 2005/0134452 A1
(43) Pub. Date: Jun. 23, 2005

(54) Implantable tracking and monitoring system

(76) Inventor: R. Scott Smith, Naples, FL (US)

Correspondence Address: Colin G. Sandercock, Esquire;Heller Ehrman White & McAuilffe LLP, 1717 Rhode Island Avenue, N.W., Washington, DC, 20036, US

(21) Appl. No.: 10/855,967

(22) Filed: May 28, 2004

Related U.S. Application Data
(63) Continuation of application No. PCT/US02/38224, filed on Dec. 2, 2002.
(60) Provisional application No. 60/334,096, filed on Nov. 30, 2001.

(51) Int. Cl. G08B 1/08

(52) U.S. Cl. 340/539.12; 340/573.1; 340/572.1; 600/300

(57) ABSTRACT

Animal and human locating devices and methods are provided that allow tracking and monitoring of lost or kidnapped children, lost elderly people, prisoners, military personnel at risk during war, and animals. The devices and techniques for their use can greatly prolong battery life and, in some cases eliminate the need of battery power altogether, allowing long term implantation. Piezoelectric power generation is described that provides long term maintenance free power through automated recharging, for both locational devices and other electronic implants in a wide range of medical technologies. The implantable devices and systems of their use can locate lost individuals or animals and also monitor their physiological status for prolonged time periods.

### FIELD OF INVENTION

The invention relates to animal tracking and monitoring systems and more particularly to devices and methods for locating wildlife, children, military troops, prisoners, aged people, and others and for automatically determining their status.

### BACKGROUND OF THE INVENTION

Human beings and other animals need to be monitored or located in a number of situations. Parents and care givers need to monitor the whereabouts of their infants and other small children and sometimes worry that a child will be kidnapped. Aged people, particularly those afflicted with Alzheimer's disease walk away and become lost, and may need monitoring for their own safety. Yet another important field for locational monitoring is for military activities. A military often has to deliver soldiers or other undercover agents into hostile territory and needs to know the location of these agents, as well as their condition. An inobtrusive locator that could be permanently placed in a soldier's body and that can report the soldier's location would provide important benefits.

Existing homing devices and other monitoring devices often suffer short-comings that make them unsuited for long range location finding needed for these instances. Two such devices that send out radio waves from inside a body are described in U.S. Pat. No. 4,262,632 issued to J. P. Hanton et al. on Apr. 21, 1981, and U.S. Pat. No. 3,034,356, issued to W. J. Bieganski et al. on May 15, 1962. These patents describe capsules that are swallowed and that transmit signals from the gastrointestinal tract. Both devices have a very limited range and are not useable for monitoring purposes. The Hanton et al. system was designed for livestock identification at a range of less than 20 feet. The Bieganski et al. device was again designed for short distance monitoring of gastrointestinal pressure.

Another device, as shown in U.S. Pat. No. 3,618,059 and issued to M. F. Allen on Nov. 2, 1971 locates personal property and packages. The device is activated by the unwanted movement of the object. This device, although valuable for tracking purposes is not small enough for convenient long term tracking of a human being.

Other devices developed for tracking and locating purposes, for example are described in U.S. Pat. No. 3,806,936 and issued to C. A. Koster on Apr. 23, 1974 concern personal locators that attach to the clothing of an individual and are activated when that person becomes lost. Although a help under some circumstances, such devices do not answer sufficiently the long term need for location finding and cannot be used easily without specific action by the user. Such devices are large, designed for short term monitoring and activation and are incompatible with continuous long term monitoring and locating. Furthermore these devices tend to have metal structures which can set off airport screening alarms and are definitely obtrusive when used for long time periods.

A smaller device that provides longer term tracking is described in U.S. Pat. No. 4,706,689 and issued to Daniel Man on Nov. 17, 1987. This device is implantable behind the ear of a human and transmits a coded signal for tracking. The device operates continuously, and requires recharging through external contacts. These features severely constrain transmission range, even when recharged daily. Further problems can arise when multiple units are used in a common area. The tracking problem becomes prohibitively expensive for many simultaneous units, and malfunction of one unit can mask signals of other units, or require significantly increased transmission power levels for all units. Moreover, this system requires an expensive closely spaced network of permanent tracking receivers with capabilities to track simultaneously multiple units.

A major problem of many homing devices and systems attempted until now is that the implanted homing unit needs to be recharged through contacts brought out through the person's skin. Such an arrangement presents a significant health hazard. Furthermore, such regular recharging significantly restrains the user's freedom, and heightens the user's awareness of the implanted device, resulting in a less free and natural state of mind. The complexity of such systems also causes false alarms, and/or prohibitively high cost.

Another example of a personal locator transmitter is shown in U.S. Pat. No. 5,014,040, issued to Weaver et al., on May 7, 1991. The personal locator transmitter discussed in this patent can be worn as wrist watch. The watch includes a manually operable alarm activated by pressing a button and an automatic alarm activated when forcibly removed from the wearer's wrist.

A common limitation of many locational devices developed thus far is their continuous activation, which severely taxes their power supplies. Generally, this problem is met by attaching the unit outside the body, where batteries can be replaced, or by recharging through electrodes that penetrate the skin. Another problem with most existing devices is that they contain significant amounts of metal and can be detected by many airport scanners. Still another problem is that many of the devices require the purchase or development of an expensive directional receiving system. Finally, most devices described thus far are bulky and not easily concealed. A user constantly is reminded of the presence of the locational device and, in many situations has to affirmatively actuate and recharge the device. A locating device that requires no particular maintenance and especially which can be forgotten by the user would be a boon to those who need to monitor such individuals as small children, aged adults and military personnel.

### SUMMARY OF THE INVENTION

Embodiments described herein provide a system that uses two way radio wave communication between an individual and a searcher, without necessarily requiring any action by the individual being searched. In many embodiments the human (or animal) may not be conscious of the radio equipment used to determine his or her location and the implanted equipment remains in a state of readiness for many years. Some embodiments utilize pre-existing electronic networks, such as cellular telephone systems, military satellites or battlefield drones to find the person or animal. In one embodiment an individual such as a parent or police officer can use equipment such as directional radio locators and signal transmitters to activate and use the locator system to find a missing child.

One embodiment of the invention provides a transponder suitable for long term implantation into a body, comprising a container with a biocompatible surface, a receiver within the container that monitors for at least one coded signal, a transmitter within the container that transmits a radio signal upon receipt of a coded signal by the receiver, a power supply that is rechargeable after implantation, and an antenna.

Another embodiment of the invention provides an animal location and monitoring system comprising at least one implantable transponder comprising a container with a biocompatible surface, a receiver within the container that monitors for at least one activation signal, a transmitter within the container that transmits a signal upon receipt of a coded signal by the receiver, a power supply that is rechargeable after implantation and an antenna, and at least one transmitter that can generate an activation signal, wherein the activation signal generated by the transmitter activates the transponder and the activated transponder then transmits a radio signal that may be used to determine the location of the transponder.

Yet another embodiment of the invention provides an electric recharger for an implantable device, comprising a sterile piezoelectric crystal coated with a biocompatible surface, the crystal having at least one vibrating plane with a dimension long enough to absorb energy after insertion into a muscle or other body part, and electrical connections for use with an implantable device.

Yet another embodiment of the invention provides a method of powering or charging an implantable electronic device, comprising supplying a sterile piezoelectric crystal coated with a biocompatible surface, the crystal having at least one vibrating plane with a dimension long enough to absorb energy after insertion into a muscle, and electrical connections for use with an implantable device, and electrically connecting the crystal of a) to the implantable device so that motion exerted onto the crystal is converted into electrical energy that powers the device.

Yet a further embodiment of the invention is a homing device suitable for long term implantation into a body, comprising a container with a biocompatible surface, a transmitter within the container that transmits a homing radio signal, and a power supply that is comprises a piezoelectric crystal that is implanted into the body and generates electricity during muscle movement.

These and other aspects of the present invention will become apparent to the skilled artisan in view of the description set forth below.

### DESCRIPTION OF FIGURES

FIGS. 1 to 3 show preferred muscles for implanting or attaching a piezoelectric generator according to embodiments of the invention.

### DETAILED DESCRIPTION OF EMBODIMENTS OF THE INVENTION

Embodiments described herein provide several useful features. One such feature is a biocompatible surface such as silastic polymer that may be used to coat the exterior of an implanted locating device, thus allowing long term implantation. Another is the use of an outside transmission signal to activate a transmitter circuit(s) for a short time and thus limit the need for an energy intensive radio transmission until desired for use by an outside receiver. This feature conveniently allows a user to set up and adjust one or more receivers for finding the homing signal at the users convenience and then instruct the implantable device to transmit a homing beacon to the waiting electronic receiver(s). By using the implantable device as a transponder, power requirements are greatly reduced and a small battery may be used over a longer time period. In further embodiments of the invention the transponder receiver circuit is kept off most of the time, further slashing power requirements.

Another useful feature provided in embodiments disclosed herein is a piezoelectric power generator that can be implanted at a suitable location in a body to generate electricity and recharge the battery. Embodiments provide piezoelectric materials of suitable shapes and dimensions that match appropriate body spaces and structures that move. The piezoelectric materials convert a portion of the absorbed mechanical energy from regular body movements into electrical energy by the twisting and/or flexing of the imbedded piezoelectric device. This feature can apply to a wide range of implantable devices, such as for example, heart pacemakers and bone growth stimulators. An embodiment of the invention provides a way to charge a small power supply such as a high capacity capacitor or a small battery from piezoelectric generated electricity.

Yet another feature that may provide added convenience and power for the implantable homing device is the combined use of the device with cellular telephone or satellite service. In this embodiment an implanted transponder transmits a signal that can be received by a cellular telephone or satellite network, allowing inexpensive and comprehensive service across much of North America or other areas of the world.

In another embodiment useful for child locating a device is programmed to stop working after a set time period. That is, a device may be implanted into a newborn or other small child and programmed to stop working what that child achieves the age of majority.

In another embodiment, the device can be temporarily or permanently deactivated. The deactivation may occur, for example by a coded instruction to the receiver, by a timer within the device that monitors the passage of a predetermined time, by magnetic force from outside that switches the unit off, by the user sending a turn off code to the unit through piezoelectric signals input to the implanted unit from voluntary muscle twitching and other ways of controlling a circuit as may be known to a skilled artisan. Such a feature could be useful, for example, with military personnel or other individuals who want the capability to readily deactivate the device, either temporarily or permanently.

Most embodiments described herein utilize an implantable transducer. The transducer preferably has several features as discussed next.

The Implantable Transponder

A transponder according to embodiments of the invention may contain a radio receiver, a radio transmitter, an antenna, a power supply and an optional case, and usually will be covered with a biologically inert material such as silastic polymer. A “container” of the transponder may be as simple as a film that covers metal wires and/or semiconductor chip or circuit board, or the optional case. The “transponder” may be as simple as a transmitter (without a receiver) but preferably both receives and transmits radio frequency energy. In a preferred embodiment the radio receiver and transmitter utilize radio frequencies above 100 megahertz and more preferably above 300 megahertz. Higher frequencies are preferred for their ability to penetrate buildings, thus facilitating detection of a homing signal at great distances. When using the highest frequencies above 100 megahertz and particularly above 300 megahertz it is preferred to locate any transmitter or receiver that communicates with the implanted transponder at a higher elevation such as on a cellular phone tower, an airplane, a hilltop or a satellite.

The Implant Transmitter

The transmitter section of the transponder may emit any type of signal that may be detected by a receiver that is useful for direction finding. For example, a complex digital signal may be used for interacting with a digital cellular telephone network. Many types of modulation and coding are known to skilled artisans such as regular tone modulated amplitude modulated, frequency modulation, and pulse modulation. More than one type of signal can be generated, preferably at different times. The signal can be continuous or of any predetermined duration, including a short duration (e.g., less than 60 seconds, less than 10 seconds, less than 2 seconds and even less than 0.5 second) to conserve battery power. The transmitter should provide the strongest radio frequency output power as possible as limited by the transmitter circuitry and the power supply. In one embodiment the transmitter outputs at least 5 milliwatts of power. In another embodiment the transmitter outputs at least 25 milliwatts of power. In yet another embodiment the transmitter outputs at least 100 milliwatts of power. In yet another embodiment the transmitter outputs at least 1 watt of power.

In yet another embodiment the transmitter outputs a very short pulse and the output power may be up to 10 watts or greater. By way of example, when a very high frequency of 1 gigahertz is used a 10 millisecond pulse of 100 watts may be achieved even by a small power supply, if suitable (e.g. very high capacitance energy storage) circuitry is used. In this case, a modulation signal may be superimposed on the signal to help a receiver discriminate the signal from background. A frequency modulation of 10 megahertz, for example imposed on the 1 gigahertz signal may allow a receiver to dectect a 10 millisecond pulse from a background noise signal. The art of radio signal generation and detection is highly developed and a skilled artisan has many alternatives for generating and detecting short coded pulses of high energy.

The Implant Receiver

The receiver section of the transponder (if used) monitors for the presence of an interrogating radio signal that preferably codes for the individual transponder and instructs that transponder to generate a homing signal. The term “monitor” here means that the receiver has at least one circuit on that is capable of responding to the presence of the interrogating radio signal. The circuit could as simple as a passive (non energy requiring) high impedance circuit that activates a receiver and/or transmitter upon detection of sufficient radio frequency energy to generate a high enough threshold voltage. In other embodiments the circuit includes one or more amplification stages and in yet further embodiments the circuit includes a passive or active filter or decoder that generates a signal when the receiver detects a coded signal.

Upon detection of a suitable signal, the receiver, in many embodiments, turns on the transmitter. The transmitter may turn on immediately, at a predetermined time, or at another time. Furthermore, the receiver itself may respond to one or more transmitted signals to turn on more frequently or for a greater duration as prompted by the presence of the signal(s). Preferably the transmitter is turned on immediately to create a homing radio signal. The receiver may operate on a convenient frequency and use signal decoding circuitry as needed to detect its specific code. This embodiment allows the use of multiple transponders in the same geographic area without extra confusion.

In a preferred embodiment the receiver is off most of the time to conserve battery power. In this case a timer may turn on the receiver periodically to check for the presence of the interrogating radio signal. An interrogating radio signal is turned on for enough time to for the receiver to hear it once during the on-off cycle. The interrogating signal may continue during this minimum time or repeat frequently during the time period until a homing signal is activated received or until a sufficient time has elapsed to order any transponder within range to send a homing signal. By way of example, a timer circuit may turn the receiver on every 10 minutes for one second. During that one second window of time the receiver monitors for and decodes any signals. If an interrogating signal is received during that one second window then the receiver activates a homing signal or signal sequence. The interrogating signal in this scenerio has to stay on or repeat itself at least every second for ten minutes or more to ensure activation of any transponders during the 10 minute timing window. This mechanism drastically slashes the long term battery requirements, which greatly increases convenience of use. This is because timing circuits generally use much less than one percent of the energy of a radio receiving circuit. By using the timer alone most of the time and turning the receiver on for one second out of every 600 seconds, a battery in standby mode can last more than one hundred times as long between charging and can conserve energy for transmitting.

The receiver and transmitter circuits may be combined in one device and may be packaged in a case. Alternatively, they may be separate. The case may be made from or covered with a biologically inert material such as Teflon or a biocompatible material consisting of silastic polymer and a polyolefin, wherein the polyolefin can be a low-density polyethylene, high-density polyethylene, linear low-density polyethylene, ultra-high molecular weight polyethylene or mixture thereof. An optional battery or large capacity capacitor, antenna and/or optional sensor(s) may be packaged together in the case or electrically connected to a circuit within the case from outside through one or more conducting leads.

The Antenna

The transmitter and receiver of the transponder typically will require an antenna to emit and absorb radio frequency energy, respectively. In one embodiment the transmitter is connected to one antenna and the receiver is connected to another antenna. This is particularly useful when the transmitter and receiver utilize frequencies that are greatly different and optionally use different size antennas. In an embodiment the receiver is disconnected from an antenna that is used also by the transmitter. The receiver further may be turned off during transmission, to prevent damage. A zener diode or other diode junction may be used instead to shunt excess radiofrequency energy that may enter the receiver during transmission and thereby protect the receiver input semiconductor junctions from high voltage burnout.

An antenna may be a short extension of the transponder circuitry, or, more preferably is a conductor that extends from the circuitry some distance such as more than 1 cm, more than 2 or even more than 5 cm. In many embodiments the antenna is made as long and/or as wide as possible, while remaining compatible with implantation into a living body, to improve the efficiency of energy transmission and/or reception. Preferably the antenna is a conductive organic material such as carbonized fiber or fabric. A conductive non-metallic material is preferred because such material is less easily detected by X-rays and other body scanners used to sense weapons.

Use of Non-Metallic Conductors

In some embodiments, metal use in the implantable transponder and antenna(s) is minimized to escape detection and/or avoid problems when the user is scanned at a security check for air transportation. For example, the implantable device can contain less than 50% by weight metal, less than 20% metal, less than 10% metal, less than 5% metal and even less than 2% metal.

The antenna can be made of a material that presents a low impedance to high frequency radio wave energy. In an embodiment the conductive material in an antenna and/or one or more circuitry components comprises at least 75% by weight organic material. In another embodiment the organic conductor comprises at least 90% of the conducting material and in another embodiment the organic conductor comprises at least 99% of the conducting material by weight. The terms “organic material” and “organic conductor” as used here refer to material that includes at least 90% by weight elements selected from the following group: carbon, sulphur, oxygen, silicon, germanium, hydrogen, nitrogen, phosphorous, and selenium. Advantageously, the organic material can comprise at least 95% by weight one or more of the listed elements. In another embodiment the organic conducting material is more than 95% by weight carbon. This use of organic material in a conductor is preferred to make the implantable device difficult to detect.

In an advantageous embodiment, the conducting material within an antenna and/or other circuit components such as contacting leads can comprise at least 75% by weight, at least 90%, or at least 99% conducting polymer(s). Conductive polymers have been studied intensively and a large variety are known. Initially polyacetylene, a conjugated organic polymer was reported as having high electric conductivity when oxidized by suitable reagents. The concept of conductivity and electroactivity of conjugated polymers was quickly broadened from polyacetylene to include a number of conjugated hydrocarbon and aromatic heterocyclic polymers, such as poly(p-phenylene), poly(p-phenylene vinylene), poly(p-phenylene sulfide), polyacetylene, polyanaline, polyquinoline, polypyrrole, and polythiophene, while success with fluorocarbon polymers was reported more recently as described in U.S. Pat. No. 6,208,075.

The principal methods for preparing conducting polymers have included electrochemical oxidation of resonance-stabilized aromatic molecules, structure modification along with doping, and synthesis of conducting transition metal-containing polymers. Each of these materials alone, in combination and also combined with metallic conductors may be used in embodiments for both the antenna(s) and/or conductive leads or other circuit components.

A useful embodiment relies on carbonized fiber or fabric made from polyacrylonitrile or other substance for the conductive material. Carbonized thread and fabric that are electrically conductive are known to the skilled artisan as, for example taught in U.S. Pat. No. 6,172,344 to Gordon et al., which is incorporated by reference in its entirety, particularly the lower half of column 6, which describes how to synthesize a fabric of organic conductor. This patent describes the heating/oxidation of polyacrylonitrile fiber. The treated fiber contains a “virtual 100% carbon content,” was finished in a fabric form of 270 gm/square meter weight and exhibited an electrical resistance at 20 degrees centigrade in the range 3-4.5 ohms per square meter across the width and 1.5 to 2.5 ohms per square meter along the length. The conductive fiber or fabric can be encapsulated or laminated with any of a range of materials to insulate it, as for example, described on column 9 and Table 1 of U.S. Pat. No. 6,172,344, which is particularly incorporated by reference.

The Power Supply

Advantageously, the power supply is a rechargeable battery such as a lithium hydride or other metal hydride battery. The battery should have an expected lifetime that exceeds the expect use of the implanted device. The battery in many embodiments will constitute the largest portion of the device and may be the heaviest element as well. The battery can be constructed as much as possible from non-metallic material. Advantageously the battery has enough capacity to transmit at least two homing pulses, and more advantageously at least 10 pulses. The battery is recharged by any of a number of techniques known to a skilled artisan.

In another embodiment the power supply is a high capacity capacitor that may be used alone as a power source or in combination with a small battery. High capacity charge storage materials have been developed for the electric car industry, some of which are available in farad sizes. These materials, in smaller packages are particularly useful for certain embodiments. In one embodiment a high power pulse of radio frequency energy is limited to a very short time to obtain a high power transmission signal. A storage capacitor is particularly suitable for supplying the short but heavy inrush of electrons through a transmitter circuit to meet this short time duration demand. After extensive or complete discharge, the capacitor may be charged up by, for example, piezoelectric generated electricity from body movements over an extended time period of hours, days or weeks. In one embodiment an implanted electronic device such as a transmitter, transponder, heart pacemaker or other device, receives at least 20%, 30%, 50%, 66%, 75%, 85%, 90%, 95%, 99% or even 100% of its power from one or more capacitors, greatly alleviating or even eliminating the need for a storage battery.

Two techniques, magnetic induction and in vivo piezoelectric generation are among the possible techniques that can be used to recharge a battery or capacitor. Magnetic induction is known and may be used where an implant recipient is able to manually hold or fasten a device such as a transformer to the outside surface of the body near the implant. U.S. Pat. No. 6,016,046 for example describes a system used for charging electric shavers through a distance, the principles of which are intended for use in this embodiment. In this case a primary coil that generates an alternating magnetic field is used outside the body and brought into proximity to (preferably into contact with) the skin near to the implanted device. The implanted device contains a secondary coil that responds to the alternating magnetic field and produces an alternating current in response thereto. The implanted device further contains an AC to DC converter and controller circuit. The rectifying and control circuit within the implanted device converts the induced electron flow into a form suitable for charging the power supply inside the body, all without electrical connection through the skin.

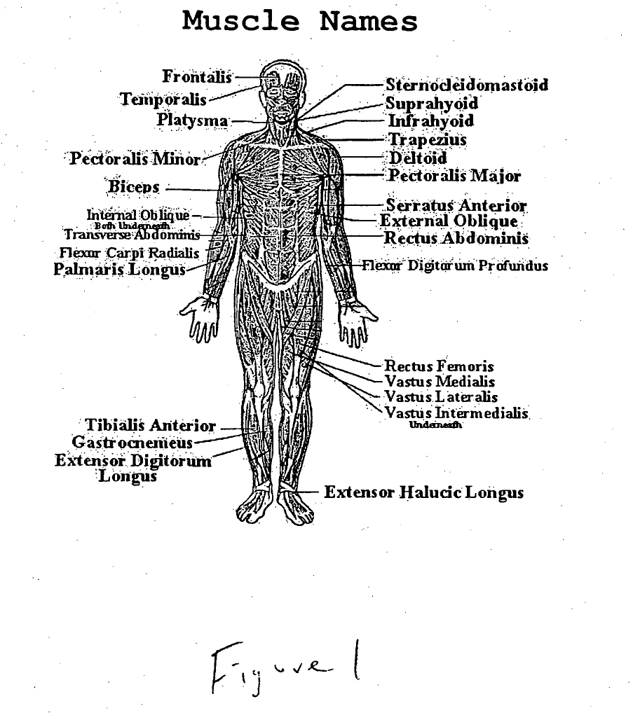

In another embodiment the implanted device further monitors the state of battery charge and signals the operator of the charging device, preferably by generation of a weak radio wave (less than 100 milliwatts, less than 10 milliwatts, or even less than 1 milliwatt) when the battery is fully charged. The charger then may stop immediately, or continue for a defined time to replenish the energy used to generate the radio wave signal. One advantage of this signaling mechanism is that the charging state signal may be formed by many of the same transmitter circuits used to create a homing signal, and thus acts as a quality assurance check that those circuits remain functional. Features of known magnetic charging devices such as the use of smoothing circuits and oscillator circuits are described, for example in FIG. 1 of U.S. Pat. No. 6,016,046 and related details from U.S. Pat. Nos. 6,316,909; 5,680,028; 5,600,225; 4,082,097 and 5,550,452, which most specifically are incorporated by reference in their entireties.

Piezoelectric Charging of Implanted Devices

In an embodiment piezoelectricity recharges a battery or capacitor automatically without conscious action on the part of the implant user. One way of carrying out this technique is with one or more piezoelectric devices that are sized and positioned within a body to capture some of the motion energy produced by the body during body movement or physiology. This embodiment utilizes the ability of certain materials to generate electricity directly upon their flexing or vibrating. The typically small current pulses generated piezoelectrically can charge a battery or capacitor within the body and thereby power a device such as a pacemaker or transponder.

In an embodiment a piezoelectric device can generate a voltage through normal movement that is more than at 100 percent, 110 percent, 120 percent, 130 percent, 200 percent 500 percent or even 1000 percent of a fully charged battery voltage. The piezoelectric device output may connect to the battery via a blocking diode to prevent battery discharge. The electrical connection(s) between the piezoelectric device and the battery are sealed to maintain high impedance and prevent loss of charging current by conductive leakage. In an embodiment the insulation resistance provides at least 1 times 10 to the 6th power ohms, at least 1 times 10 to the 7th power ohms, at least 1 times 10 to the 8th power ohms or at least 1 times 10 to 9th ohms. In one embodiment the piezoelectric device is connected to a capacitor, which stores the charge until needed.

Many types of piezoelectric materials are known that generate electricity and are useful for embodiments, such as, for example, discussed in U.S. Pat. No. 4,387,318 issued to Kolm et al.; U.S. Pat. No. 4,404,490 issued to Taylor et al.; U.S. Pat. No. 4,005,319 issued to Nilsson et al. and U.S. Pat. No. 5,494,468 issued to Demarco, Jr. et al. Certain piezoelectric materials are particularly well suited that comprise polymers which can be cast in the form of plastic sheets or other forms. Particularly, polymers known as PVDF polymers are contemplated. The term “PVDF” means poly vinylidene fluoride. The term “PVDF polymer” means either the PVDF polymer by itself and/or various copolymers comprising PVDF and other polymers, e.g., a copolymer referred to as P(VDF-TrFE) and comprising PVDF and PTrFE (poly trifluoroethylene).

Many PVDF polymers are known and have been commercialized for example, as dielectric materials for capacitors. Although these materials, as commonly used, have no or minimal piezoelectric properties, such properties are added by a technique called “poled.” By “poled” is meant that electric dipoles in the materials, which normally randomly orient, become aligned. Alignment may be carried out by heating to enhance dipole mobility and applying a relatively large D.C. voltage, which aligns the dipoles along electrostatic field lines provided by the voltage. The materials are cooled and, when the dipole mobility is low, the voltage is removed, which permanently freezes the aligned dipoles in place.

PVDF polymers are commercially available as sheets and may be formed to include thin electrodes of various metals, e.g., silver, aluminum, copper and tin, as well as known conductive inks or organic polymer on their opposite major surfaces. The sheets are relatively strong and tear resistant, flexible and chemically inert. Such PVDF polymer piezoelectric materials may be inserted as, for example, long pieces aligned along an axis of movement within or next to a body structure such as a muscle that periodically or occasionally moves. When flexibility is desired for a region of the a piezo element that is connected to a conductor for a charging circuit, the metal electrode(s) if used may be made from metal(s) of high ductility, e.g., tin and silver, and a known conductive ink including, for example, carbon black or silver particles.

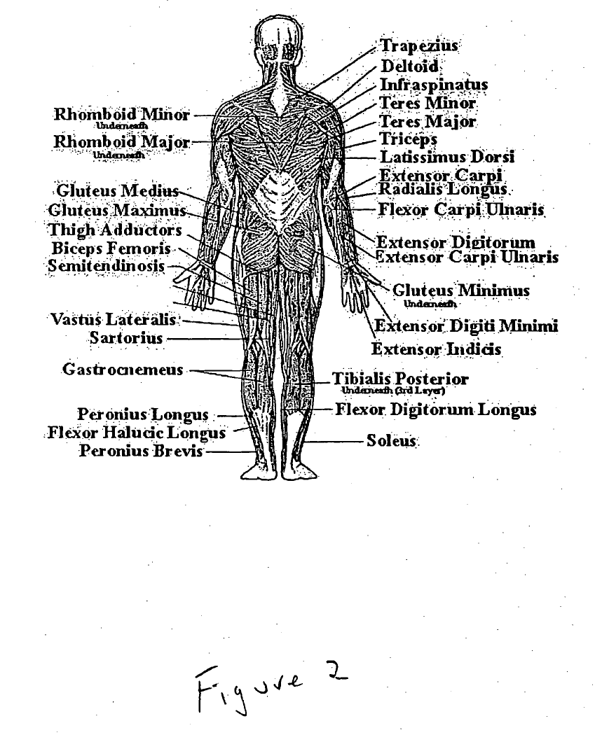

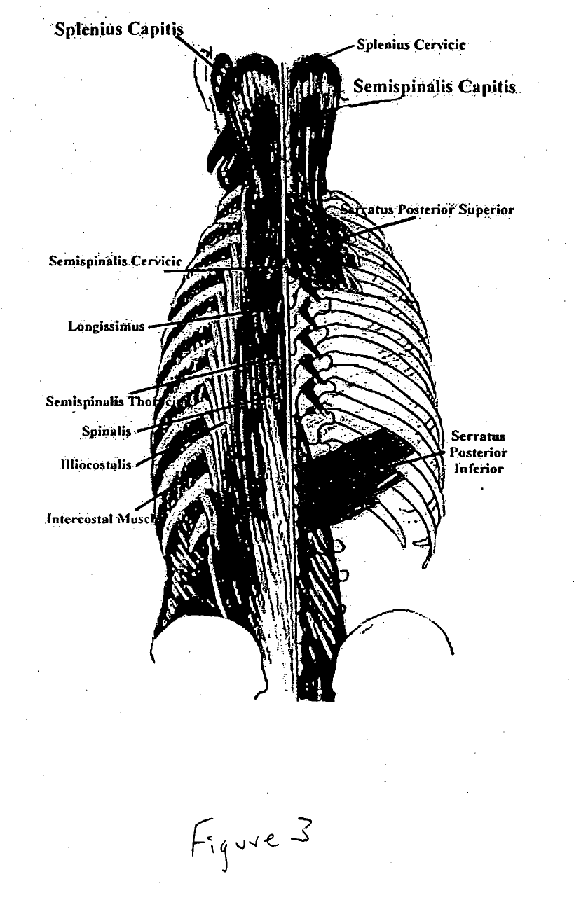

Certain regions and spaces in an animal body such as a human, dog, cat, circus animal or zoo animal are more ideal for placement of a piezoelectric charger than others. In an embodiment a piezoelectric device is inserted into a muscle. In the case of a human, one or more muscles preferably are selected from the group consisting of a gastronemius muscle, a soleus muscle, a forearm muscle, biceps muscle, neck muscle, abdominal muscle, or other muscle as, for example listed in FIGS. 1 to 3. The piezoelectric device and insulated conducting leads to it can be implanted in a muscle away from sensory nerve locations.

Other non-muscle structures also may be used to activate a piezoelectric device after attaching or embedding device in the structure. An abdominal wall or chest wall may be used by the device. One type of piezoelectric device is a cable or thin ribbon that may be embedded parallel to a long axis of the muscle or non-muscle structure. Another type of device is a flat sheet, which optionally is positioned on the exterior surface of the muscle or non-muscle structure. In one embodiment a flat piezoelectric device is adhered to a muscle or other structure by a chemical adhesive, biological adhesive, or mechanical coupling such as one or two sutures. A piezoelectric device may be coated with one or more polymers to insulate the device electrically. In one embodiment the device coating has an outer layer that adheres particularly well to biological structures, such as, for example, a polycationic coating, which binds well to anionic surfaces of the body, or a “molecular velcro” such as that described in WO 0032542 by Battersby et al.

In another embodiment a piezoelectric device in a thin ribbon form between 0.5 and 10 cm long and between 0.01 cm and 0.2 cm wide is implanted into a right gastronemius muscle away from the Achilles tendon. Insulated carbonized conducting fiber is used to connect the piezoelectric device to a transponder that is positioned between the muscle and fat layer of the right buttocks. The transponder contains a very small battery and capacitor that can power a radio frequency transmission from the transponder.

An advantageous feature of piezoelectric power generation in the body is that the piezoelectric elements can be fashioned in a wide variety of shapes and sizes both to match appropriate moving structures in the body and to escape detection by regular X-ray devices and other scanners. In this context it is particularly desirable to combine plastic piezoelectric power generator device(s) with non-metallic conductors. These structures may be electrically isolated from surrounding physiological fluid by a coating, e.g., of polymer such as a silastic polymer, a multiple polymer coat such as silastic polymer on a base of other rigid plastic, or other arrangement, as for example shown in U.S. Pat. No. 6,172,344.

In one embodiment a piezoelectric crystal element is hermetically sealed. Advantageously the crystal element itself and/or a hermetic covering of the element is coated with a biocompatible material such as a silastic polymer. Other FDA accepted materials may be used as well. In an embodiment the sealing material presents a high resistance of a minimum value as described above to prevent leakage of current from the piezoelectric device.

Sensors for the Transponder

A transponder according to embodiments described herein may provide more information beyond a homing signal. The transponder may encode a signal, by for example, modulation, timing, or selection of frequency to indicate a physiological state or change in state. The transponder may include for example, a thermister or other temperature monitoring device, a shock or vibration sensor, a pressure sensor, a tilt sensor to indicate whether the individual is lying down for example, a light sensor to detect whether the subject is in a dark room, or a conductivity sensor to detect whether the device has been removed from the individual.

Miniature sensors are well known in electronics and a wide range are contemplated for embodiments of the invention. Preferably a sensor is within or closely located near the transponder circuitry but may arise from a property of another element such as an antenna, power lead, or piezoelectric generator. For example, a power lead between a circuit and a battery or charger circuit or antenna may be made from a non-metallic conductor such as carbon fibre as described elsewhere herein. Such lead has a temperature coefficient of conductivity, which can be monitored to determine temperature. In this case the lead serves a dual purpose as electricity conductor and temperature sensor.

A piezoelectric device for charging batteries directly generates an electrical signal with frequency and amplitude characteristics that correspond with body movements and is particularly useful as a sensor. Abrupt or certain types of body movements can be sensed directly and notice of them sent via the transmitter outside the body without the use of an additional sensor. A light sensor that operates in the deep red region (preferably between 600 nanometer to 1200 nanometers, more preferably between 690 to 900 nanometers) can sense light that transmits through skin. Such sensor can detect relative lighting conditions outside the body. This information can be transmitted.

A tilt sensor may be included which determines whether an axis of the transponder is horizontal or vertical, and report this information for transmission. Sensed information preferably is sent by the transmitter via a single transmission. This embodiment is particularly useful for military monitoring of personnel who may be captured and held in unknown locations or conditions. The transponder can inform by radio transmission not only the location of a captured individual but also whether the person is sitting or lying down, in a dark room or not, is moving or not and is dead or not.

In another embodiment a tilt sensor is used to improve the signal from a transponder, greatly increasing the transponder range. A problem addressed by this embodiment is that high frequency communications tend to be directional and are sensitive to antenna orientation. Using the tilt sensor, a transponder may sense whether its antenna is vertical or horizontal and may wait to send a signal until a desired antenna orientation occurs. In some cases this will be the horizontal orientation. In a military application the transponder may even alert the user that the transponder has been asked to send a transmission, so the user can shift his body for optimal transmission in consideration of the antenna orientation. In yet another embodiment the transponder signals the user that a rescue is imminent and to prepare for rescue. In these situations the transponder may signal the user by creating an electric shock or vibration within the user's body.

In yet another embodiment an individual with an implant uses a piezoelectric sensor of the implant to transmit a signal when desired. In this case, the individual may flex a muscle or muscle group that contains one or more voluntary muscle, at a particular rhythm or cadence, triggering the transponder to transmit a signal. The transponder may respond to the piezoelectric pulses and transmit a signal when the particular voltage pattern is sensed.

Transponder Implantation and Use

A transponder according to certain embodiments may be placed virtually anywhere in the body. Preferably the transponder is inserted between a muscle and a fat layer. Most preferably the transponder is inserted into deep soft tissue. Plastic surgeons are familiar with areas of superficial fascia, such as loose connective tissue containing mostly fat deep to the skin as interwoven fibers intermingled with occasional elastic fibers in spaces that are occupied by fat cells and which can accomodate and hide a transponder. Other spaces consist of fat around organs and that lining the abdominal cavity. Deep fascia, or fat that adheres closely with muscle also are useful areas, particularly in combination with a piezoelectric power generator that may be implanted in the adjoining muscle layer. Any other area of subcutaneous adipose tissue which may occur in the face, legs, arms, abdomen, and or buttocks also is a particularly good candidate for receiving a transponder.

The magnetic induction charging device or piezoelectric device, if used may be implanted along with the transponder and used to maintain the power supply of the transponder. More advantageously, a piezoelectric device constantly provides some charging, and may be used with a large control circuit and input capacitance for charging the battery without overcharging. As reviewed above the transponder may inform a receiver of the transponder's status or battery status. If necessary the transponder and/or power source can be removed for repair or replaced by a simple operation. When used as a child locator this equipment preferably is implanted early in the child's life. In this case the transponder can have a timer that automatically inactivates the transponder at a predetermined time, such as when the child becomes 16 or 18 years old.

A transmitter and receiver outside the body are used to activate the transponder and detect a homing signal respectively. In this case both the receiver and transmitter of the transponder are used to communicate with transmitter(s) and receiver(s) outside of the body. In another embodiment the transponder automatically sends a homing signal according to a predetermined time or when energy is available. In such case the transponder may not include a receiver portion but only a transmitter portion. In a preferred embodiment a transmitter outside of the body sends an activation signal, which the transponder receives. Upon receipt of the activator signal the transponder activates the transmitter to send a pulse. The pulse is detected by one or more receivers located outside the body at a distance therefrom. The receiver(s) typically are located more than 10 meters, often more than 100 meters or even kilometers away from the transponder. The receiver can have a directional antenna and determines the direction of the homing pulse. Advantageously, two or more receivers detect the homing pulse and create information that allow the determination of the transponder location in two or three dimensional space.

Another embodiment uses a cellular telephone service as a receiving grid. In this embodiment the transponder may generate a homing signal on its own or after prompting by an activating signal. The signal may come from a portable transmitter, a drone aircraft, a satellite, a commercial radio station, a cellular telephone service, or other source. When using the cellular telephone service, preferably the transponder generates a signal in a form for contacting a particular call-in number that has been established for the purpose of finding missing persons. In one embodiment the cellular telephone service grid records the received homing signal from two or more antennas. The homing signal may be received either at the same time or sequentially by the two or more antennas and the cell phone grid provides this more detailed information about the location and/or relative movement of the individual with the implanted transponder.

All above cited references, patents and patent applications are hereby incorporated in their entireties by reference.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention. In the claims which follow, the articles “a”, “and”, “the” and the like shall mean one or more than one unless indicated otherwise.

1. A transponder suitable for long term implantation into a body, comprising:
a) a container with a biocompatible surface;
b) a receiver within the container, that monitors for at least one coded signal;
c) a transmitter within the container, that transmits a radio signal upon receipt of a coded signal by the receiver;
d) a power supply that is rechargeable after implantation; and
e) an antenna.

2. The transponder of claim 1, wherein the biocompatible surface is a silastic polymer.

3. The transponder of claim 1, wherein the antenna of e) is an elongated organic conductor that extends outside of the container.

4. The transponder of claim 1, wherein the power supply of d) is recharged by at least one of piezoelectric generation of energy through mechanical body movement acting upon one or more implanted piezoelectric crystals, and magnetic induction through an alternating magnetic field external to the body acting upon an inductor in the transponder.

5. The transponder of claim 1, wherein the signal transmitted by the transmitter of c) is of a suitable frequency and coding to activate a cellular telephone network.

6. The transponder of claim 1, further comprising a conductivity testing circuit that detects when the transponder is removed from the body.

7. The transponder of claim 1, wherein the transponder further comprises a timer that inactivates the transponder at a given time.

8. The transponder of claim 1, wherein the receiver of b) turns on periodically to monitor for the coded signal and upon detection of that signal, activates the transmitter of c) to emit a location signal.

9. The transponder of claim 8, wherein the receiver of b) turns on for an interval of less than 60 seconds at least once every hour.

10. The transponder of claim 9, wherein the receiver of b) turns on for an interval of less than 2 seconds at least once every 30 minutes.

11. The transponder of claim 1, wherein the transponder comprises at least 85 percent non-metallic material.

12. The transponder of claim 1, further comprising at least one sensor selected from the group consisting of a temperature sensor, a tilt sensor, a pressure sensor, a shock sensor, and a light sensor, and wherein the transponder transmits sensed information upon the sensor output exceeding a preset limit, or upon command by a coded turn on signal.

13. A device for finding and monitoring a living animal, comprising a container with a biocompatible surface, a receiver within the container that monitors for at least one activation signal, a transmitter within the container, that transmits a signal upon receipt of a coded signal by the receiver, a power supply that is rechargeable after implantation into the animal and an antenna.

14. An animal location and monitoring system comprising:
a) at least one implantable transponder comprising a container with a biocompatible surface, a receiver within the container, that monitors for at least one activation signal, a transmitter within the container, that transmits a signal upon receipt of a coded signal by the receiver, a power supply that is rechargeable after implantation and an antenna;
b) at least one transmitter that can generate an activation signal, wherein the activation signal generated by the transmitter activates the transponder, the activated transponder then transmits a radio signal that may be used to determine the location of the transponder.

15. The system of claim 14, wherein the biocompatible surface is a silastic polymer.

16. The system of claim 14, wherein the antenna of e) is an elongated organic conductor that extends outside of the container.

17. The system of claim 14, wherein the power supply of d) is recharged by at least one of piezoelectric generation of energy through mechanical body movement acting upon one or more implanted piezoelectric crystals, and magnetic induction through an alternating magnetic field external to the body acting upon an inductor in the transponder.

18. The system of claim 14, wherein the signal transmitted by the transmitter of c) is of a suitable frequency and coding to activate a cellular telephone network.

19. The system of claim 14, further comprising an impedance monitoring circuit that detects when the transponder is removed from the body and triggers the transmitter to send a signal upon the removal.

20. The system of claim 14, wherein the transponder further comprises a timer that inactivates the transponder at a given time.

21. The system of claim 14, wherein the receiver of b) turns on periodically to monitor for the coded signal and upon detection of that signal, activates the transmitter of c) to emit a location signal.

22. The system of claim 21, wherein the receiver of b) turns on for a short interval of less than 60 seconds at least once every hour.

23. The system of claim 21, wherein the receiver of b) turns on for a short interval of less than 60 seconds at least once every hour.

24. The system of claim 14, wherein the transponder comprises at least 85 percent non-metallic material.

25. The system of claim 14, further comprising at least one sensor selected from the group consisting of a temperature sensor, a tilt sensor, a pressure sensor, a shock sensor, and a light sensor, and wherein the transponder transmits sensed information upon the sensor output exceeding a preset limit, or upon command by a coded turn on signal.

26. The system of claim 14, wherein at least two transponders are implanted at secret locations within the body of a human.

27. The system of claim 14, wherein at least one transponder is implanted under the skin of an animal.

28. The system of claim 14, wherein the transmitter of b) is on a satellite or aircraft.

29. An electric recharger for an implantable device, comprising a sterile piezoetectric crystal coated with a biocompatible surface, the crystal having at least one vibrating plane with a dimension long enough to absorb energy after insertion into a muscle or other body part, and electrical connections for use with an implantable device.

30. A method of powering or charging an implantable electronic device, comprising:
a) supplying a sterile piezoelectric crystal coated with a biocompatible surface, the crystal having at least one vibrating plane with a dimension long enough to absorb energy after insertion into a muscle, and electrical connections for use with an implantable device; and
b) electrically connecting the crystal of a) to the implantable device so that motion exerted onto the crystal is converted into electrical energy that powers the device.

31. A homing device suitable for long term implantation into a body, comprising:
a) a container with a biocompatible surface;
b) a transmitter within the container that transmits a homing radio signal; and
c) a power supply that is comprises a piezoelectric crystal that is implanted into the body and generates electricity during muscle movement.

32. The homing device of claim 31, further comprising one or more sensors for detecting and reporting a condition, selected from the group consisting of a thermister or other temperature monitoring device, a shock or vibration sensor, a pressure sensor, a tilt sensor to indicate whether the individual is lying down for example, a light sensor to detect whether the subject is in a dark room, and a conductivity sensor to detect whether the device has been removed from the individual.
